(19) United States
(12) Patent Application Publication
(10) Pub. No.: US 2026/0069819 A1
(43) Pub. Date: Mar. 12, 2026

(54) Electronic Emotional Support Device

(71) Applicant: Big World, Denver, CO (US)

(72) Inventor: Delaney Maeve Larmor, Denver, CO (US)

(73) Assignee: Big World, Denver, CO (US)

(21) Appl. No.: 19/323,980

(22) Filed: Sep. 9, 2025

Related U.S. Application Data
(60) Provisional application No. 63/692,391, filed on Sep. 9, 2024.

(51) Int. Cl. A61M 21/00

(52) U.S. Cl. CPC A61M 21/00; A61M 2021/0022; A61M 2021/0027; A61M 2021/005; A61M 2205/505; A61M 2205/52; A61M 2205/8237

(57) ABSTRACT

A handheld, electronic device and method for providing emotional support to a live being is disclosed. The device comprises programmable operations stored in a memory, processed by a processor, and displayed on a graphical display. Emotional and biological data may be input into the device and saved. Based on the programmed operations a response to the data is supplied via the graphical display. Further, data sets including date and time are added to the input data and stored in the memory for future viewing. The programmable operation may display a group of input data on the device display and/or connect to another device comprising the programmed operation for processing and display of the input or reaction data. The device may comprise AI and/or machine learning programs to suggest helpful information or tasks to the user upon processing data and input into the device.

The following application is a U.S. application for patent under 35 USC 111 (a). The application claims priority to U.S. Patent application No. 63/692,391 filed Sept. 9, 2024.

### FIELD

This disclosure relates to the field of psychological health, especially an electronic emotional device for supporting emotional health in real-time with artificial intelligence and/or machine learning enabling improved feedback.

### BACKGROUND

Real-time support of psychological and/or emotional health can be a crucial component for well-being. Unfortunately, support professionals or other support people can't always be available when a person is undergoing psychological or emotional stress and/or emotions. Further, if sought, such ongoing care can be expensive and time intensive. There is a need for real-time, quality emotional and/or psychological support that improves quality of life.

### SUMMARY

Because health professionals and other support people can't always be available when a person is undergoing psychological or emotions stress and/or emotions, there is a need in the field for a system to provide real-time psychological and/or emotional support.

The present disclosure comprises a device and method for providing emotional support, mood lifting, and improving mental health. In one aspect, the device comprises an electronic device for supporting psychological and/or emotional health of a user, the device comprising: a touchscreen display; at least one processor; a memory unit; a power supply means; at least one selection button; a vibrating motor; sound producing system; wherein the at least one processor comprises programming for at least one support module, wherein the processor gives direction to the touchscreen display to display components of the support module; wherein the at least one processor receives, and memory unit saves, to the appropriate at least one support module, at least one user status; and at least one of an artificial intelligence (AI) or machine learning program that prompts the user to take an action based on the at least one user status.

The device comprises at least one support module wherein the support module(s) are programmed software to receive, store, and display data related to at least one of a journal support module, a to-do list support module, a game support module, a focus-timer support module, a calendar support module, a music support module, a notes module, and a settings support module. The at least one support module may also be selected from at least one of a mood tracker, a community journal, a goal tracker, a points tracker, and an electronic sticker sharing and collage module. The at least one of an artificial intelligence (AI) or machine learning program collects multiple of the at least one user status and attaches an incident with each of the at least one user status, wherein the at least one of an artificial intelligence (AI) or machine learning program establishes at least one pattern of a behavior associated with the at least one user status and the incident and prompts the user with a direction to improve a symptom from at least one of the at least one support module The at least one support module displays at least one of a pop-up message, a graphic, or suggestion for action from one of the at least one support module(s) in response to the at least one user status. The user may create an image of a representative character. Each of the at least one support module(s) incorporates the created image of the representative character in at least one display graphic.

The device may further comprise a means for connecting to a cloud environment or a separate communication enabled processing device. The electronic device may connect to a software application processed from at least one of a cloud environment or a separate communication enabled processing device comprising the at least one support module. the software application is capable of at least one of receiving, storing, and displaying data from the electronic device on a communication enabled processing device. The processor of the electronic device receives information from the software application and displays the information on the touchscreen display of the electronic device.

The disclosure further comprises a method for improving emotional and/or psychological health of a user, the method comprising: providing an electronic device, the electronic device comprising: a touchscreen display; a processor; a memory unit; a power supply means; a vibrating motor; sound producing system; wherein the processor directs the touchscreen display to display at least one support module; and wherein the processor receives and memory saves at least one user status; and inputting, by the user, at least one user status; and inputting, by the user, at least one user status. The method may further comprise providing a selection of characters to act as a representative character. The method may further comprise providing a selection of anxiety relieving exercise prompts. The method may further comprise providing a system for inputting and storing feeling data. The method may further comprise providing a system for inputting and storing journal information, wherein the journal information may be selected from at least one of a body image log, a feeling log, a food log, and an exercise log. The method may further comprise wherein a support module comprises a focus timer. The method may further comprise wherein a support module comprises a system for playing one of sounds and music. The method may further comprise comprising providing a support module for one or more of the following including a to-do list, one or more games, a calendar, note taking, settings, and AI learning.

### BRIEF DESCRIPTION OF THE DRAWINGS

FIG. 1 is a front view of an electronic support device of the present disclosure.

FIG. 2 is a top side view of the electronic support device of the present disclosure.

FIG. 3 is a back side view of the electronic support device of the present disclosure.

FIG. 4 is a left side view of the electronic support device of the present disclosure.

FIG. 5 is a right side view of the electronic support device of the present disclosure.

FIG. 6 is a bottom side view of the electronic support device of the present disclosure.

FIG. 7 is a depiction of the interior of the electronic support device of the present disclosure.

FIG. 8 is a block diagram illustrating the function of the electronic support device.

FIG. 9 is a block diagram illustrating a method for supporting a user of the present disclosure.

FIGS. 10-13 are screen shots of application functions of the electronic support device.

FIGS. 14-18 are screen shots of application functions of the electronic support device.

FIGS. 19-26 illustrate application functions of the electronic support device.

FIGS. 27-28 illustrate the note taking function of the electronic support device.

FIGS. 29-30 illustrate the music application of the electronic support device.

FIGS. 31-33 illustrate the calendar application of the electronic support device.

FIG. 34 illustrates a timer function of the electronic support device.

FIGS. 35-37 illustrate a body journal application of the electronic support device.

FIGS. 38-39 illustrate a productivity application of the electronic support device.

FIG. 40 illustrates a game application of the electronic support device.

FIG. 41 illustrates an example environment of the electronic support device.

Before explaining the disclosed embodiments of the present disclosure in detail, it is to be understood that the invention is not limited in its application to the details of the particular arrangement shown, since the disclosure is capable of other embodiments. Also, the terminology used herein is for the purpose of description and not of limitation.

### DETAILED DESCRIPTION

The present disclosure comprises a device and method for providing emotional support, mood lifting, and improving mental health. In one aspect, the device comprises an electronic device for supporting psychological and/or emotional health of a user, the device comprising: a touchscreen display; at least one processor; a memory unit; a power supply means; at least one selection button; a vibrating motor; wherein the at least one processor comprises programming for at least one support module, wherein the processor gives direction to the touchscreen display to display components of the support module; wherein the at least one processor receives, and memory unit saves, to the appropriate at least one support module, at least one user status; and at least one of an artificial intelligence (AI) or machine learning program that prompts the user to take an action based on the at least one user status.

The device comprises at least one support module wherein the support module(s) are programmed software to receive, store, and display data related to at least one of a journal support module, a to-do list support module, a game support module, a focus-timer support module, a calendar support module, a music support module, a notes module, and a settings support module. The at least one support module may also be selected from at least one of a mood tracker, a community journal, a goal tracker, a points tracker, and an electronic sticker sharing and collage module. The at least one of an artificial intelligence (AI) or machine learning program collects multiple of the at least one user status and attaches an incident with each of the at least one user status, wherein the at least one of an artificial intelligence (AI) or machine learning program establishes at least one pattern of a behavior associated with the at least one user status and the incident and prompts the user with a direction to improve a symptom from at least one of the at least one support module The at least one support module displays at least one of a pop-up message, a graphic, or suggestion for action from one of the at least one support module(s) in response to the at least one user status. The user may create an image of a representative character. Each of the at least one support module(s) incorporates the created image of the representative character in at least one display graphic.

The device may further comprise a means for connecting to a cloud environment or a separate communication enabled processing device. The electronic device may connect to a software application processed from at least one of a cloud environment or a separate communication enabled processing device comprising the at least one support module. the software application is capable of at least one of receiving, storing, and displaying data from the electronic device on a communication enabled processing device. The processor of the electronic device receives information from the software application and displays the information on the touchscreen display of the electronic device.

The disclosure further comprises a method for improving emotional and/or psychological health of a user, the method comprising: providing an electronic device, the electronic device comprising: a touchscreen display; a processor; a memory unit; a power supply means; a vibrating motor; wherein the processor directs the touchscreen display to display at least one support module; and wherein the processor receives and memory saves at least one user status; and inputting, by the user, at least one user status; and inputting, by the user, at least one user status. The method may further comprise providing a selection of characters to act as a representative character. The method may further comprise providing a selection of anxiety relieving exercise prompts. The method may further comprise providing a system for inputting and storing feeling data. The method may further comprise providing a system for inputting and storing journal information, wherein the journal information may be selected from at least one of a body image log, a feeling log, a food log, and an exercise log. The method may further comprise wherein a support module comprises a focus timer. The method may further comprise wherein a support module comprises a system for playing one of sounds and music. The method may further comprise comprising providing a support module for one or more of the following including a to-do list, one or more games, a calendar, note taking, settings, and AI learning.

### Terminology

As used herein, conditional terms including but not limited to “can”, “could”, “might”, or “may”, unless expressly stated otherwise, or otherwise understood within the context of use, are generally meant to mean that certain examples include, while other examples do not include, particular features, steps, and/or elements. These terms is not generally meant to imply that features, steps, and/or elements are required for one or more examples or that one or more examples necessarily includes logic for deciding, with or without user input or prompting, whether these features, steps, and/or elements are included or performed in any particular examples.

As used herein, conjunction language such as the phrase “at least one of X, Y, and Z”, unless otherwise stated, is understood to mean that an item, term, or element, etc., may be either X, Y, or Z, or any combination thereof. It is not generally intended to imply that certain examples require at least one of X, at least one of Y, and at least one of Z to be present. Use of the phrase, “at least one of X, Y, or Z, as used in general to mean that an item, term, or element, etc., may be either X, Y, or Z, or any combination thereof.

As used herein, the word “or” in reference to a list of two or more items includes all the following interpretations: any one of the items in the list, all the items in the list, and any combination of items in the list. Likewise, the term “and/or” in reference to a list of two or more items, includes all the following interpretations: any one of the items in the list, all the items in the list, and any combinations of items in the list.

As used herein, the terms “comprise”, “comprises”, and “comprising” are to be construed as inclusive terms, as opposed to exclusive or exhaustive, meaning “including, but not limited to.”

As used herein, “connected” or “coupled”, or any variant thereof, means any connection or coupling, either direct or indirect between two elements which may be physical, logical, or a combination thereof.

As used herein, the words “herein”, “above”, or “below”, and similar words, when used in the application, refer to the application as a whole and not to a single section of the application.

Computer programs generally comprise one or more instructions, or set of instructions, placed at various times in various memory devices of a computing device, which, when read and executed at least one processor, causes the computing device to carry out the functions involving the disclosed techniques. In some examples, a carrier containing the one or more instructions, or set of instructions is provided. The carrier may comprise an electronic signal, an optical signal, a radio signal, or a non-transitory computer readable storage medium.

Any one of or all of the aforementioned features may be combined with each other, except where otherwise stated or wherein the combination may be incompatible due to their function or structure, or apparent to persons skilled in the art. Unless not physically possible, it is envisioned that methods and steps described herein may be performed in any sequence and/or combination, and the components may be combined in any functional manner.

In some examples, certain algorithms, events, acts, directions, or functions of any of the algorithms, steps, functions, processes, or programs described herein can be performed in a different order, and may be added, merged, or left out (e.g., not necessary for the practice of the algorithm(s)). In some examples, certain algorithms, events, acts, directions, or functions of any of the algorithms, steps, functions, processes, or programs described herein can be performed concurrently through, for example, multi-threaded processing, interrupt processing, multiple processors or processor cores or on other parallel architectures, rather than sequentially.

Systems and modules described herein may comprise software, firmware, or hardware, or combination(s) thereof, suitable for the purposes described herein. Software and other modules may reside and execute on servers, workstations, personal computers, computerized tablets, PDAs, and other computing devices suitable for purposes described herein.

Software and other modules described herein may be accessible via local computer memory, via a network, via a browser, and/or via any other means appropriate for purposes described herein. Data structures described herein may comprise computer files, variables, programming arrays, programming structures, or any electronic information storage schematics or methods, or any combination thereof, appropriate for purposes described herein.

User interface may comprise features from graphical user interfaces, interactive voice response, command line interfaces, and/or other appropriate interfaces.

Various components, features, and/or elements described herein may be implemented in one or more virtual environments or machines or an isolated execution environment, rather than on a dedicated computer hardware system and/or computing device.

Data repositories may represent physical and/or logical storage including for instance, storage area networks or other distributed storage systems.

Connections between elements or steps shown represent possible paths of data flow, rather than actual connections between hardware. While some examples of connections are shown, any of the subset of elements can communicate with any other subset of elements in various implementations.

### Description

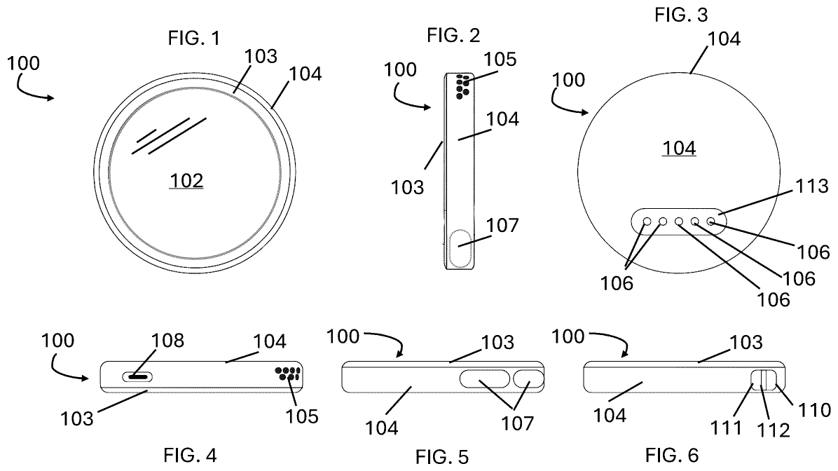

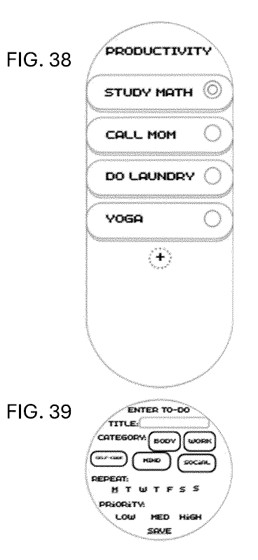

As illustrated in detail in FIGS. 1-7, an example electronic emotional support device 100 comprises a device as shown, illustrated, and detailed in FIGS. 1-41. Specifically, as shown FIGS. 1-6, the electronic emotional support device 100 comprises a display screen 102, a front housing 103, a back housing 104, wherein the front housing 103 and back housing 104 may be a single piece formed for example from injection molding or other known methods of manufacture. In alternate embodiments, the front housing 103 and back housing 104 may be two separate pieces fitted together by known methods. The back housing 104 is shown as enclosing the sides of the device 100, however alternate designs are possible wherein the front housing 103 encloses the sides of the device 100, or wherein the front housing 103 and back housing 104 meet in the middle of the sides. The front housing 103 and back housing 104 may be formed from a plastic, being, for example, a polycarbonate or thermoplastic polyurethane material. The display screen 102 may, for example, be a liquid crystal display (LCD) or light-emitting diode (LED) display of which various types are known including but not limited to organic LED, quantum dot LED, MicroLED, and electronic paper (E-Ink).

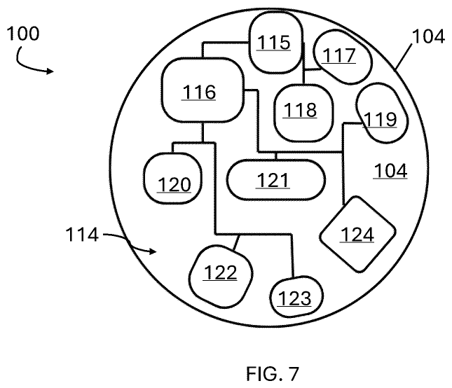

FIG. 2 illustrates a top view of the electronic emotional support device 100 including the front housing 103, back housing 104, a speaker 105, and a toggle button 107. The toggle button 107 functions to turn the device on and off, allow entry of data, or entry of a command to at least one processor shown in FIGS. 7, 116. FIG. 3 illustrates a back view of the electronic emotional support device 100 including the back housing 104, one or more prongs 106 for contacting a charging and/or powering device (not shown), and a prong housing 113 to support and retain the prongs 106 in the back housing 104. The prongs 106, being a metal or metal alloy, or other material suitable for relaying electrical current, are shown for illustration purposes. Varying number of prongs 106 may be used to accommodate various designs of charging and/or powering devices.

FIG. 4 illustrates a left side view of the electronic emotional support device 100, including the speaker 105, and an UBC charging/connecting component 108. FIG. 5 illustrates a right side view of the electronic emotional support device 100 including two toggle buttons 107 used for input of commands or data. In an example one toggle button 107 positioned above the other toggle button 107 may be used for input of a yes or affirmative command or to move a cursor up on the display screen 102. A lower toggle button 107 may be used to input a no or negative command or to move a cursor down on the display screen 102. Various functions for the toggle buttons 107 may be used or programmed for the device 100. FIG. 6 illustrates a bottom view of the electronic emotional support device 100 including an attachment means 110 that comprises a depression 111 in the back housing 104 and a bar 112 spanning the depression, wherein a cord, lanyard, or clip (not shown) is threaded through under the bar 112 in the depression 111 or attached to the bar 112.

FIG. 7 illustrates an internal compartment 114 of the electronic emotional support device 100. The front housing 103 is removed to illustrate the internal components of the internal compartment 114. The interior of the back housing 104 is illustrated and serves as medium for adhering internal components thereto. Internal components include but are not limited to components needed to power and drive the electronic and software systems and applications of the electronic emotional support device 100. The placement of the components is not binding as other designs may be applicable. Lines between the components illustrate connectedness of the components and systems. Wiring and/or other connection components are not shown but are including in the disclosure as necessary components for operation of the electronic emotional support device 100.

As illustrated in FIG. 7, a vibrator 115 may be located and/or positioned near a wall of the back housing 104. The at least one processor 116, button components 117, 119, speaker components 118, wireless communication chip 120, power system 121, speaker 122, alternate charging components 123, such as electrical components to support charging via UCB-C or the like charging devices, and a memory card 124 are housed in the internal compartment 114. Alternate components may include a microphone and recording system for intake and recording of sound.

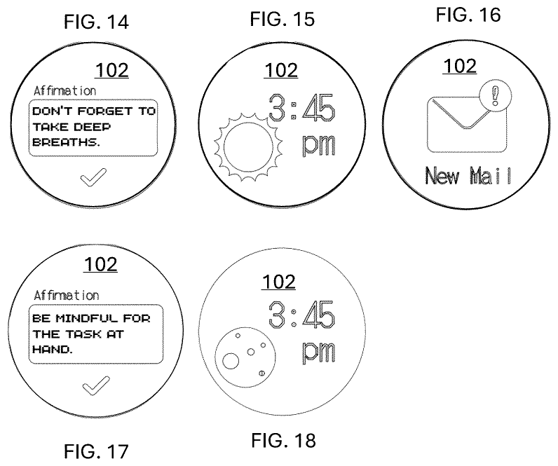

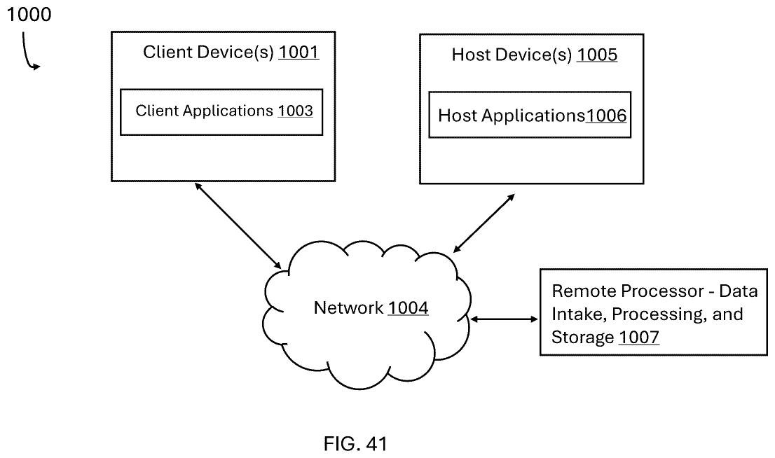

In an example the electronic emotional support device 100 comprises at least one processor 116 that is a microcontroller unit (MCU) processor. In some aspects, the electronic emotional support device 100 comprises at least one processor that is a system on a chip (SoC) processor. In some examples, the electronic emotional support device 100 collects data from the systems and applications wherein data from each of the systems is compiled over time and, via the at last one processor 116 or at least one separate connected processor FIGS. 41, 1105, the data, along with other user data, is used to detect trends in order to create an on-going treatment plan for managing and improving emotional and/or psychological well-being. Artificial intelligence (AI) and/or machine learning (ML) software programs may be implemented by the at least one processor 116 or at least one separate connected processor FIGS. 41, 1105 which may be a host device(s) 1005 with host applications 1006 connected through a network 1004, being wireless. Alternately AI and ML functions may be processes in a cloud based system being one example of a connected network FIGS. 41, 1004, or by another separate remote processor 1007 used for data intake, processing, and storage. The electronic emotional support device 100, being a client device FIGS. 14, 1001 and it's client applications or associated software 1003, may be processed and housed in the electronic emotional support device 100, by a host device 1005 and its applications 1006, by a cloud network 1004, or by a remote processor 1007.

Further examples of processors 116 include but are not limited to a microcontroller unit (MCU) processor, also termed a microcontroller, which is a compact computer on a single chip that integrates a central processing unit (CPU), being a processor, memory, and input/output peripherals to control one or more specific function(s) in an embedded system. Unlike microprocessors, which are just a CPU, MCUs contain all the necessary components to run a program and operate a device. Another example of a processor that may be suitable for use in the user-wearable device 100 of the present disclosure includes a system on a chip (SoC) processor, being a complete computer system integrated onto a single integrated circuit or chip. It combines various components like the CPU, GPU, memory, and other peripherals onto one piece of silicon. This integration offers benefits like reduced size, lower power consumption, and improved performance compared to traditional multi-chip systems. In a non-limiting example, the at least one processor 116 may comprise an Espressif Systems ESP32-S3 being a low-power MCU-based system on a chip (SoC) with integrated 2.4 GHz Wi-Fi and Bluetooth®.

As illustrated in FIG. 7, a wireless communications chip 120 provides the electronic emotional support device 100, with capability to send and receive data to and from the at least one processor 116 or other electronic systems. A wireless communication system 120 in a preferred embodiment may comprise a wireless communication chip 120 being an integrated circuit that enables devices to send and receive data without physical cables, acting as a bridge between radio, cellular, Bluetooth® or other wireless signals and a device's digital electronics. These communications chips 120 may include radio frequency (RF) transceivers, signal processors, and power management components to support various protocols like Wi-Fi, Bluetooth®, Bluetooth® Low Energy (BLE) signal, NFC, or near field communication, a short-range wireless technology that enables communication between two electronic devices within a few centimeters of each other, and cellular signals. Other components to support powering, communication, and function of the systems not limited to wiring and other components that are necessarily included in the interior compartment 114 of the device 100 as are required for design considerations may not be shown but would necessarily be included. Other examples of connectivity technologies include but are not limited to Low Energy (Bluetooth® LE) consisting of high-performance dual-core microprocessor (Xtensa® 32-bit LX7), a low power coprocessor, a Wi-Fi baseband, a Bluetooth® LE baseband, RF module, and numerous peripherals.

As illustrated in FIG. 7, the memory card 124, may be at least one memory card 124, and may serve as sole memory, or additional memory capacity for the electronic emotional support device 100. The memory card(s) 124 may include but are not limited to a standard capacity (SD) card, SD high capacity (SDHC) card, and SD extended capacity (SDXC) which may have memory capacity from 2 GB to 2 TB. Alternate memory cards include the microSD that may also require an SD adapter.

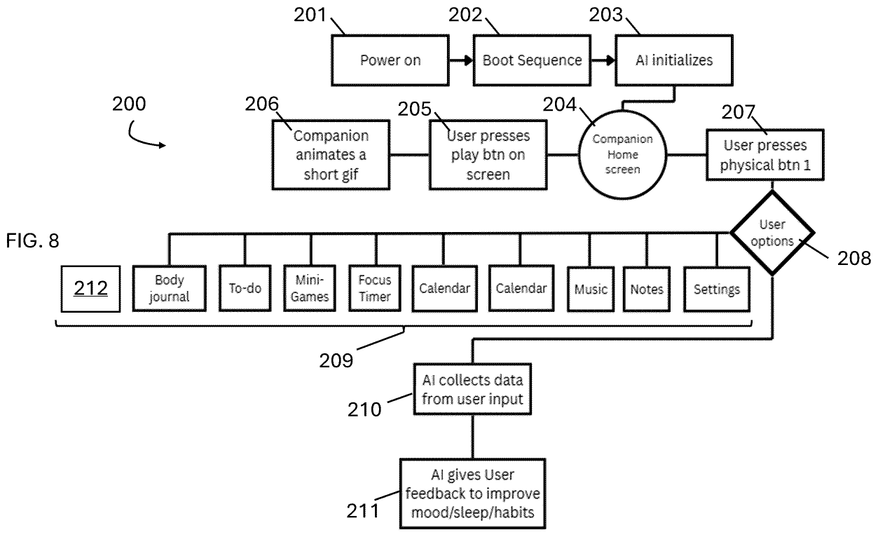

FIG. 8 illustrates method of use, applications, and capabilities 200 of the electronic emotional support device 100. A user, being a live person, powers on 201 the device, wherein the device may undergo a boot sequence 202 that initiates systems of the device. The AI/ML component of the software program of the processor also initializes 203. A companion home screen 204 may be displayed wherein the user presses a play 205 or initiate button. For purposes of the present disclosure, “companion” or “companion program” refers to the software programming or coding including the AI/ML functions of the electronic emotional support device 100. The processor may run the companion program to present an animated gif 206 on the display screen. The user may press the upper button 207, also known as physical button 1 to open a user options menu 208. User options or companion application/systems, being software support modules 209, include but may not be limited to a body journal, which records physical or emotional feelings of the user, diet, and exercise, a to-do list, games or mini-games, a focus timer, at least on calendar, music storage, notes, and settings applications. Additional support modules 212 include but are not limited to at least one of a mood tracker, a community journal, a goal tracker, a points tracker, and an electronic sticker sharing and collage module.

The user inputs data into or interacts with these support modules 209 and the at least one processor 116 that includes the AI/ML programming collects data from the user input and/or interactions 210 that may be stored on the electronic emotional support device 100 or in a separate physical electronic device or cloud software system as illustrated in FIG. 41. Following data input and/or collection the AI/ML software applications of the processor 116 may correlate patterns of improved emotional well-being based or action and/or input of the user. Following this pattern correlation, the AI/ML software application may give the user feedback to improve a mood, sleep, or habits of the user with the goal of improving over emotion and/or psychological well-being of the user 211. The AI/ML software applications of the processor 116 may continue to refine suggestions or feedback to the user to continually improve well-being, repeating steps 210 and 211 in an on-going manner. In other words, the artificial intelligence (AI) and/or or machine learning program collects multiple of a user status or input and attaches an incident with each of the at least one user status, wherein the at least one of an artificial intelligence (AI) or machine learning program establishes at least one pattern of a behavior associated with the at least one user status and the incident and prompts the user with a direction to improve a symptom from at least one of the at least one support module.

In other processes the support modules 209, 212 displays one a pop-up method, a graphic, or suggestion for action in response to a user status or input. Other support modules functions may include providing a selection of anxiety relieving exercise prompts, inputting and storing feeling data, providing a system for inputting and storing journal information, wherein the journal information may be selected from at least one of a body image log, a feeling log, a food log, and an exercise log, and/or providing a support module for one or more of the following including a to-do list, one or more games, a calendar, note taking, settings, and AI learning. Examples of these displays can be seen in FIGS. 10-40. Also as illustrated in FIGS. 10-40, the user may create an image of a representative character, wherein each of the at least one support module(s) 209, 212 incorporates the created image of the representative character in at least one display graphic.

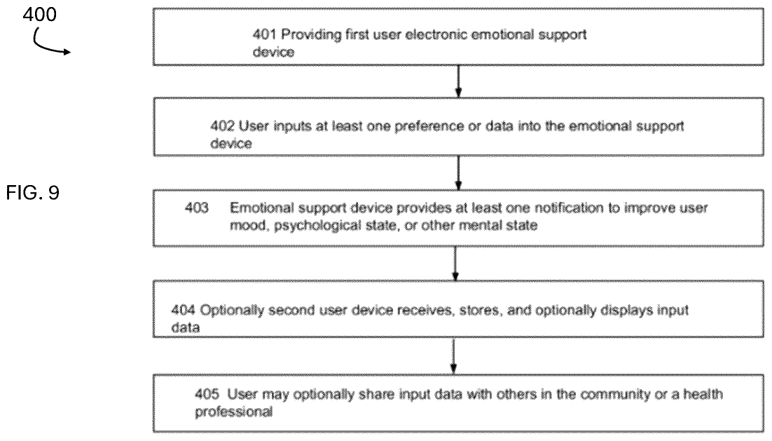

FIG. 9 is a flow chart illustrating a method of improving a mood, emotional, or psychological state of a user comprising of the present disclosure. A first step 401 comprises providing a first user with an electronic support device 100, described herein. A second step 402 comprises inputting, by the user, at least one preference or data, or interacting with the device 100 in some way including but not limited to playing games of the system, inputting calendar events, logging body data including diet, exercise, mood, using the focus timer, inputting to-do items, listening to music, logging notes, and/or other applications that may be contemplated that may improve mood and overall psychological well-being of the user, one example being a mediation application. A third step 403 comprises displaying by the electronic emotional support device 100 at least one notification, in the form of a word or words, image, animation, sounds, and/or vibrations, to improve user mood, psychological, or other mental state, in one example being suggestions for example breathing exercises, meditation, focus, presenting an appropriate song from the music application, an affirmation, or the like. Optionally a second connected host device, or host, in an example being a support or medical professional device system, may receive, store, and optionally display input data 404. In another optional step, the user may share input data with other users in the community or a health professional 405.

FIG. 41 is a block diagram of an example networked electronic emotional support device 100, being a client device 1001 with client application(s) 1003 being support modules 209 housed either on a user/client device 1001 as described herein or the electronic emotional support device 100 as described herein in FIGS. 1-40, and environment 1000, in accordance with the disclosure presented herein. FIG. 41 represents one example of a networked computer environment or system 1000 and other embodiments and arrangements are possible. The networked computer environment comprises 1000 one or more computing devices. These one or more computing devices may comprise any combination of hardware and software configured to implement the various logical components described herein. For example, one or more computing devices may include one or more memoires to store instruction for implementing the various components described herein, one or more hardware processors configured to execute the instructions stored in the one or more memories, and various data repositories in the one or more memories for storing data structures utilized and manipulated by the various components.

In some examples, as illustrated in FIG. 41, one or more user or client devices 1001 are coupled to each other, or host devices 1005 via one or more networks 1004. Networks 1004 broadly represent one or more LANs, WANs, cellular networks (e.g., LTE, HSPA, 3G, 4G, 5G, and other cellular technologies), and/or networks using any wired, wireless, terrestrial microwave, or satellite links, and may include public Internet, being cloud or other networks. These networks 1004 may employ other physical remote processor(s) 1007, memory units, for data intake, processing, and storage. One or more host devices 1005 may be employed by, for example, a support professional or medical professional, who views and tracks user data as granted access by the user. Host applications 1006, on the host device 1005, comprising at least one processor, memory, and other components required, may receive data and operate AI/ML and other software processes and support modules 209 to provide feedback to the user through the electronic emotional support or client device 100 and its client applications 1003.

In the illustrated example, a network computer environment 1000 includes one or more host devices 1005. Host devices 1005 may include, but are not limited to, any number of computers, virtual machine instances, and/or data centers that are configured to host or execute one or more instances of the host application(s) 1006. For purposes of the present disclosure, one or more host devices 1005 may be involved, directly or indirectly, in processing requests received from client devices 1001 via a network 1004. Each host device 1005 may comprise, for example, one or more of a network devices 1007, a web server, an application server, a database, and other components required to process the client device request. One or more host devices 1005 may be configured and host applications 1006 may be configured to implement the network-based or cloud-based 1004 application.

Optionally, client devices 1001 communicate with one or most host devices 1005 or applications 1006 to exchange information. The communication between the client device 1001 and the host application 1006 may, for example, be based on Hypertext Transfer Protocol (HTTP) or any other network protocol. Content delivered from the host application 1006 to the client device 1001 may include HTML documents, media content, or the like. Communication between a client device 1001 and the host application 1006 may include sending data files or packages and/or various requests and receiving data files or packages. In some examples, host device applications 1006 may host or execute the client device applications except in a remote fashion on the host device 1005 or on other network, being cloud-based processors 1004 or remote processors 1007.

Client devices 1001 represent the example electronic emotional support device 100 optionally capable of interacting with other user emotional support device(s) 1001 via a network 1004, of other users, and/or of the same user being laptops, computers, smartphones, tablets, or other devices running applications, systems, and methods of the electronic emotional support device 100. In this manner, data and applications of the electronic emotional support device 100 may be viewed, and/or shared, and interacted with on other connected devices when the electronic emotional support device 100 may not be physically present via an app or application software. Examples of other client devices 1001 include, without limitation, the disclosed client device 100, described herein, which may optionally communicate with other client devices 1001 including smart phones, tablet computers, handheld computers, wearable devices, laptop computers, desktop computers, servers, portable media players, gaming devices, and so forth. In general, client devices 1001 can provide access to different content, for instance, content provided by one or more host devices 1005. Each client device 1001 may comprise one or more user or client applications 1003 being programmed support modules and related functions 209 as described herein.

In some examples, each client device 1001 may host or execute one or more user applications 1003 described herein that are capable of interacting with one or more client devices 1001, or optionally a host device 1005, via one or more networks 1004. For instance, a user application 1003 may be or comprise an application software program or other software program stored on the device or remotely on a host device 1005. For example, a user application 1003 may comprise a mobile application or “app”. In an example, an operator of a network-based service hosted by one or more host devices 1005 may make available one or more mobile apps that enable users of client devices 1001 to access various resources of the network-based service.

In some examples, data intake, processing, and storage may take place on a remote processor 1007 and may be achieved by alternate non-user or non-host servers, processors, and memory units, whether connected directly or indirectly, wirelessly or wired. These data intake, processing, and storage services and applications may be cloud supported or may be retained in a physical device such as in examples of user or host devices described herein.

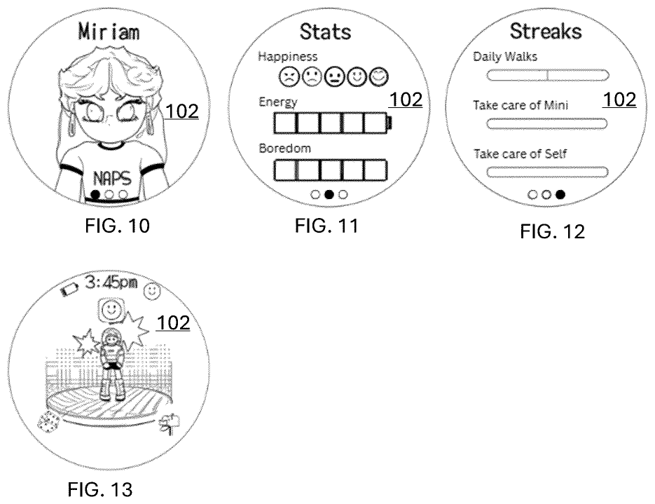

FIGS. 10-13 illustrate various graphic displays created by the electronic emotional support device 100 applications support modules 209 and shown on the display screen 102. As illustrated in FIG. 10, the user may create a companion or avatar representation of themselves. In the example shown in FIG. 10, the companion is named Miriam. As illustrated in FIG. 11, statistics or “stats” may be displayed, selected, and/or input, via the touch-enabled display screen 102. As illustrated in FIG. 12, streaks, or continuous days of performance, may be tracked and displayed including but not limited to exercise, for example, daily walks, taking care of the “Mini”, being the companion or avatar of the electronic emotional support device 100, or self-care tracking. As illustrated in FIG. 13, a badge may indicate level of performance via either interaction with the device, improved mood, or other indicators.

FIG. 14-18 illustrate various graphic displays created by the electronic emotional support device 100 support modules 209 and shown on the display screen 102 that are prompted by the AI/ML software application on the processor. These include, but are not limited to, as are seen in FIG. 14 affirmations, FIG. 15, spirit-lifting icon feedback, FIG. 16, notifications for e-mail, FIG. 17, suggestions for affirmations, reminders, and recommendations, and FIG. 18, that displays a graphic perhaps for calming.

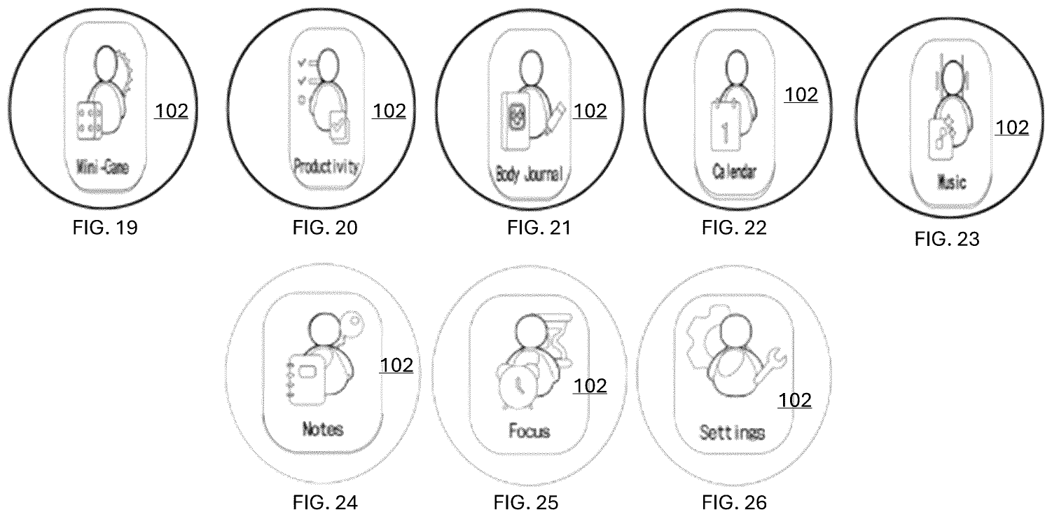

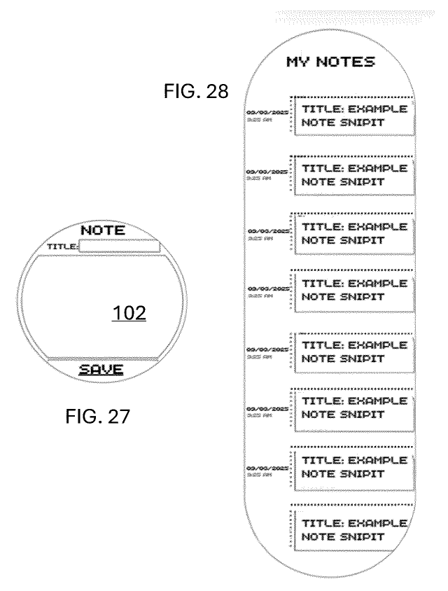

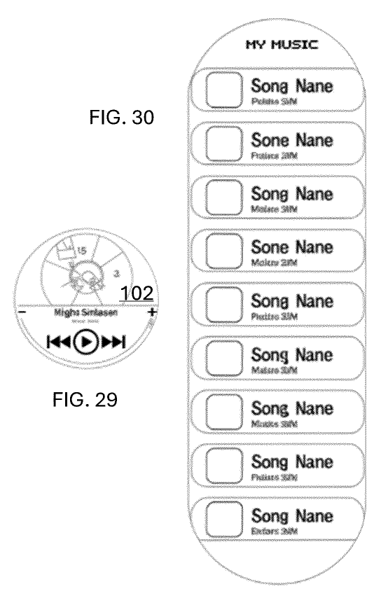

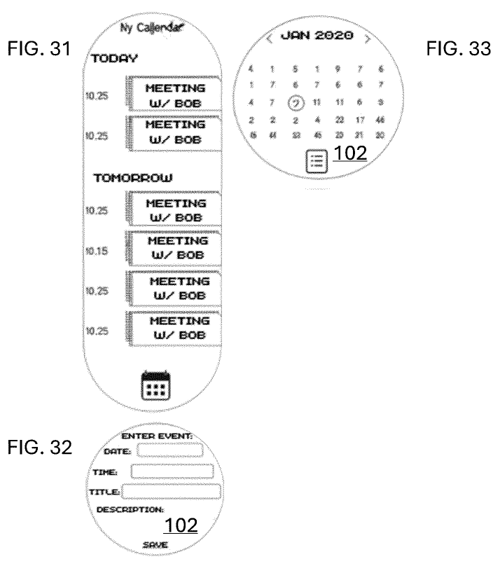

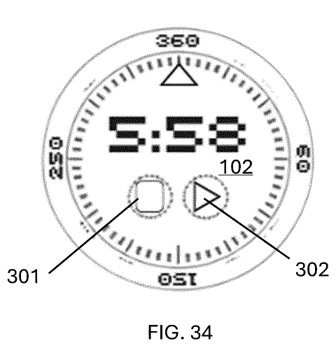

FIGS. 19-26 illustrate example functional applications or support modules 209 programmed in the processor 116 of the electronic emotional support device 100. These include at least games or mini-games, a productivity tracker, a body journal, a calendar, a music application, a notes application, a focus application, and a settings application. FIG. 27 illustrates a screen shot of the notes support module 209 and the form displayed for entering and saving a note to the support module software and processor 116. FIG. 28 illustrates a drop down or scroll menu that may be scrolled through to view all notes entered in the notes support module 209. FIG. 29 illustrates a display for a song selection in the music support module 209. FIG. 30 illustrates a drop down or scroll menu that may be scrolled through to view all songs entered in the music support module 209. FIG. 31 illustrates a drop down or scroll menu for the calendar support module showing calendar events entered into the calendar support module 209. FIG. 32 illustrates a display screen 102 view of creating a calendar entry. FIG. 33 illustrates a display screen 102 view of the calendar support module 209 display. FIG. 34 illustrates a display screen 102 view of the focus support module 209 displaying a time that can be paused by tapping the display screen 102 over the square pause button 301, or started via tapping the display screen 102 over the forward arrow button 302.

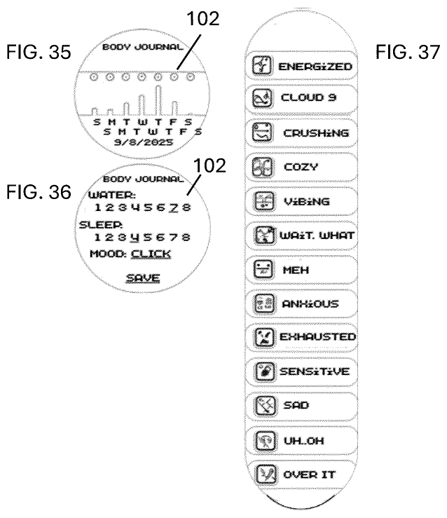

FIGS. 35-37 illustrate another example support module 209 programmed on the processor 116 of the electronic emotional support device 100, the body journal support module 209. As illustrated in FIG. 35 the application may display a bar graph of performance over time, days, weeks, months, or years. As illustrated in FIG. 36, a user may input water consumption, sleep, and mood to the body journal support module 209. FIG. 37 illustrates a drop-down menu of example mood choices that a user may select in the body journal support module 209.

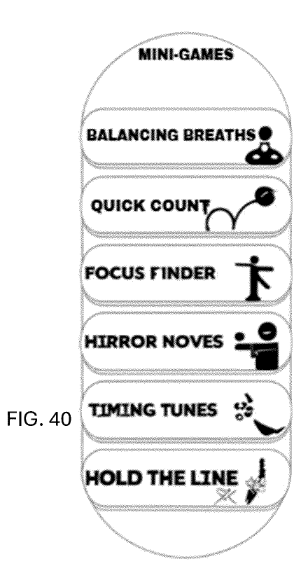

FIG. 40 illustrates a drop-down menu of games or mini-games that are programmed into the electronic emotional support device 100 as a support module 209. Example games may include but are not limited to balancing breaths, quick count, focus finder, mirror moves, timing tunes, and a hold the line game. These games are designed to lower stress and anxiety levels, increase focus, and/or other goals that increase emotional well-being at the time of playing.

Although the present device and method has been described with reference to the disclosed embodiments, numerous modifications and variations can be made and still the result will come within the scope of the disclosure. No limitation with respect to the specific embodiments disclosed herein is intended or should be inferred. Each apparatus embodiment described herein has numerous equivalents.

What is claimed is:

1. An electronic device for supporting psychological and/or emotional health of a user, the device comprising:
a) a touchscreen display;
b) at least one processor;
c) a memory unit;
d) a power supply means;
e) at least one selection button;
f) a vibrating motor;
g) sound producing system;
h) wherein the at least one processor comprises programming for at least one support module, wherein the processor gives direction to the touchscreen display to display components of the support module;
i) wherein the at least one processor receives, and memory unit saves, to the appropriate at least one support module, at least one user status; and
j) at least one of an artificial intelligence (AI) or machine learning program that prompts the user to take an action based on the at least one user status.

2. The electronic device of claim 1, wherein the at least one support module is a programmed software to receive, store, and display data related to at least one of a journal support module, a to-do list support module, a game support module, a focus-timer support module, a calendar support module, a music support module, a notes module, and a settings support module.

3. The electronic device of claim 2, wherein the at least one support module may also be selected from at least one of a mood tracker, a community journal, a goal tracker, a points tracker, and an electronic sticker sharing and collage module.

4. The electronic device of claim 1, wherein the at least one of an artificial intelligence (AI) or machine learning program collects multiple of the at least one user status and attaches an incident with each of the at least one user status, wherein the at least one of an artificial intelligence (AI) or machine learning program establishes at least one pattern of a behavior associated with the at least one user status and the incident and prompts the user with a direction to improve a symptom from at least one of the at least one support module.

5. The electronic device of claim 1, wherein the at least one support module displays at least one of a pop-up message, a graphic, or suggestion for action from one of the at least one support module(s) in response to the at least one user status.

6. The electronic device of claim 1, wherein the user may create an image of a representative character.

7. The electronic device of claim 6, wherein each of the at least one support module(s) incorporates the created image of the representative character in at least one display graphic.

8. The electronic device of claim 1 further comprising a means for connecting to a cloud environment or a separate communication enabled processing device.

9. The electronic device of claim 8, wherein the electronic device may connect to a software application processed from at least one of a cloud environment or a separate communication enabled processing device comprising the at least one support module.

10. The electronic device of claim 9, wherein the software application is capable of at least one of receiving, storing, and displaying data from the electronic device on a communication enabled processing device.

11. The electronic device of claim 9, wherein the processor of the electronic device receives information from the software application and displays the information on the touchscreen display of the electronic device.

12. A method for improving emotional and/or psychological health of a user, the method comprising:
providing an electronic device, the electronic device comprising:
a touchscreen display;
a processor;
a memory unit;
a power supply means;
at least one selection button;
a vibrating motor;
sound producing system;
wherein the processor directs the touchscreen display to display at least one support module; and
wherein the processor receives and memory saves at least one user status;
inputting, by the user, at least one user status; and
displaying on the electronic device at least one of a graphic, word, phrase, and sound to provide emotional and/or psychological support by the processor.

13. The method of claim 12 further comprising providing a selection of characters to act as a representative character.

14. The method of claim 12 further comprising providing a selection of anxiety relieving exercise prompts.

15. The method of claim 12 further comprising providing a system for inputting and storing feeling data.

16. The method of claim 12 further comprising providing a system for inputting and storing journal information.

17. The method of claim 16, wherein the journal information may be selected from at least one of a body image log, a feeling log, a food log, and an exercise log.

18. The method of claim 12, wherein in a support module comprises a focus timer.

19. The method of claim 12, wherein a support module comprises a system for playing one of sounds and music.

20. The method of claim 12 further comprising providing a support module for one or more of the following including a to-do list, one or more games, a calendar, note taking, settings, and AI learning.
